(19) United States
(12) Patent Application Publication
(10) Pub. No.: US 2022/0168337 A1
(43) Pub. Date: Jun. 2, 2022

(54) PRODUCING ATP AND IMPROVING MITOCHONDRIAL FUNCTION IN A MAMMAL USING A POLY-OXYGENATED METAL HYDROXIDE

(71) Applicant: HEMOTEK, LLC, Frisco, TX (US)

(72) Inventors: John W. Woodmansee, JR., Frisco, TX (US); Brooks Bash, Estero, FL (US); Bjorn Song, Cockeysville, MD (US)

(21) Appl. No.: 17/674,728

(22) Filed: Feb. 17, 2022

Related U.S. Application Data
(63) Continuation-in-part of application No. 17/027,516, filed on Sep. 21, 2020, now abandoned.
Application No. 17/027,516 is a continuation-in-part of application No. 16/405,287, filed on May 7, 2019, now abandoned.
Application No. 16/405,287 is a continuation-in-part of application No. 15/983,922, filed on May 18, 2018, now Pat. No. 10,278,988.
Application No. 15/983,922 is a continuation-in-part of application No. 15/797,972, filed on Oct. 30, 2017, now abandoned.
Application No. 15/797,972 is a continuation of application No. 15/346,549, filed on Nov. 8, 2016, now Pat. No. 9,801,906.
Application No. 15/346,549 is a continuation-in-part of application No. 15/183,403, filed on Jun. 15, 2016, now Pat. No. 9,649,335.
(60) Provisional application No. 62/315,524, filed on Mar. 30, 2016.
(60) Provisional application No. 62/824,912, filed on Mar. 27, 2019.

(51) Int. Cl. A61K 33/08; A61K 47/69; A61K 47/02; A61K 9/00; A61K 9/08; A61K 33/06; A61K 47/68; A61K 47/60

(52) U.S. Cl. CPC A61K 33/08; A61K 47/6923; A61K 47/6921; A61K 47/02; A61K 47/60; A61K 9/08; A61K 33/06; A61K 47/6823; A61K 9/0026

(57) ABSTRACT

A method of treating a mammal, including a human, comprising administering a therapeutically effective amount of a poly-oxygenated aluminum hydroxide composition to the mammal to improve mitochondrial function and efficiency. The poly-oxygenated aluminum hydroxide composition causes increased production of adenosine triphosphate (ATP) in the mammal. The poly-oxygenated aluminum hydroxide composition comprises a clathrate containing bioavailable pure (100%) oxygen gas (O₂) molecules that are freely released to the mammal depending on the oxygen demand, i.e., more O₂ molecules released in hypoxic regions. The administration can be oral and the bioavailable O₂ molecules are time released into the mammal. The O₂ bioavailability of poly-oxygenated aluminum hydroxide composition can be slow if the mammal, organ or tissue is well perfused and oxygenated, but rapid if hypoxic.

### CLAIM OF PRIORITY

This application is a Continuation-in-Part (CIP) of U.S. patent application Ser. No. 17/027,516 entitled Improving Mitochondrial Function in a Mammal using a Poly-Oxygenated Metal Hydroxide filed Sep. 21, 2020, which is a Continuation-in-Part (CIP) of U.S. patent application Ser. No. 16/405,287 entitled A. POLY-OXYGENATED METAL HYDROXIDE AND CBD filed May 7, 2019, which is a Continuation-in-Part (CIP) of U.S. patent application Ser. No. 15/983,922 entitled REDUCING THE PROLIFERATION OF CARCINOMA CELLS BY ADMINISTRATION OF A POLY-OXYGENATED METAL HYDROXIDE, which is a Continuation-in-Part (CIP) of U.S. patent application Ser. No. 15/797,972 filed Oct. 30, 2017, entitled REDUCING THE PROLIFERATION OF CARCINOMA CELLS BY ADMINISTRATION OF A POLY-OXYGENATED METAL HYDROXIDE, which is a Continuation-in-Part (CIP) of U.S. patent application Ser. No. 15/183,403 filed Jun. 15, 2016, entitled INTRAVENOUS ADMINISTRATION OF AN OXYGEN-ENABLE FLUID, which claims priority 01:U.S. Provisional Patent Application Ser. No. 62/315,524 entitled OXYGEN ENABLED RESUSCITATIVE FLUID filed Mar. 30, 2016, the teachings of which are incorporated herein by reference in their entirety.

### TECHNICAL FIELD

This disclosure is directed to composition and method of improving mammalian body function.

### BACKGROUND

Dermal wounds are a seemingly inevitable element of today's world. Injury to skin occurs regularly in everyday life and can otherwise be inflicted by a number of medical procedures. The vast majority of these wounds are classified as acute and will heal within several weeks of injury, however chronic wounds can take years to heal and are associated with a number of complications. Typically, wound healing is characterized by three overlapping, continuous stages: inflammation, proliferation, and wound remodeling. Within each of these stages, there is complex system of coordinating mechanisms that ultimately leads to the closure of the site of injury; each of these phases have been determined to be heavily dependent on the presence or absence of oxygen.

Oxygen is a fundamental building block in tissue repair. It functions as a nutrient, antibiotic, supports angiogenesis, cell motility, and extracellular matrix formation. Conversely, hypoxic conditions generally impair wound healing. However, the relationship between wound healing and oxygen is not a simple one and has been discussed and debated in numerous studies. For example, the initiation of wound healing is said to be stimulated by hypoxia. The inflammatory phase is dependent upon reactive oxygen species (ROS), whose activity are initiated by an absence of oxygen. ROS are considered critical to wounds at low concentrations as they are capable of stimulating growth factors and angiogenesis, acting as scavengers to destroy bacteria, and debriding damaged tissue. However, as hypoxia onsets, the production of ROS becomes increasingly improbable due to a lack of available oxygen available for creating the compounds. In combination with increasing hypoxia, a lack of ROS prevents wounds from advancing through subsequent stages of wound healing causing them to become infected or chronic. In general, as tissue repair progresses, the demand for oxygen increases and the supply decreases. This crisis in the availability of oxygen is due to metabolic processes consuming large amounts of the resources as they attempt to repair the wound site. This explains why supplemental oxygen delivery to the wound site is vital and why many studies have attempted to fill this therapeutic gap in wound healing technologies.

Chronic wounds are a major target for medical technological development. In the United States, there are 6.5 million patients affected by chronic wounds each year with an estimated $25 billion spent annually on their treatment. Chronic wounds are defined as being arrested in one of the stages of wound healing, usually the inflammatory or proliferative phase. Typically, a wound becomes chronic in the presence of foreign material, bacteria, or pathogens which invoke the production of cellular constituents and impede wound healing by using or destroying building blocks such as oxygen, causing the wound to remain hypoxic. A supply of oxygen to wounded tissue via microcirculation is critical for both wound healing and resistance to infection. Chronic wounds are particularly compromised in this regard and therefore require supplemental oxygen administration in order to heal. As such, the administration of supplemental oxygen has shown significant beneficial impact on the treatment of chronic wounds by providing cells with sufficient oxygenation for progression through subsequent wound healing phases.

### SUMMARY

A method of treating a mammal comprising administering a therapeutically effective amount of a poly-oxygenated aluminum hydroxide composition to the mammal to improve mitochondrial function and efficiency, wherein the poly-oxygenated aluminum hydroxide composition comprises a clathrate containing bioavailable free oxygen gas (O₂) molecules. The poly-oxygenated aluminum hydroxide generates adenosine traphosphate (ATP) in the mammal, including a human.

### BRIEF DESCRIPTION OF THE DRAWINGS

FIG. 1 illustrates a method of intravenously administering a mammal a therapeutically effective amount of a poly-oxygenated metal hydroxide in accordance with this disclosure;

FIGS. 2A-2D are diagrams illustrating systemic characteristics of 50% isovolemic hemodilution, including hematocrit, heart rate, mean arterial pressure, and pulse pressure. Measurements were taken immediately prior to (BL) and following (HD t0) hemodilution;

FIG. 3A shows tissue oxygenation (P_{ISF}O₂) following 50% volume replacement using Ox66™ in a crystalloid. All P_{ISF}O₂ values (mmHg) were normalized to baseline (BL) for ease of comparison;

FIG. 3B shows tissue oxygenation (P_{ISF}O₂) following 50% volume replacement using Ox66™ in a crystalloid, using particles smaller than those in FIG. 3B, and further shows tissue oxygenation using PEGylated Ox66™ particles in a Colloid;

FIG. 3C shows survival results of specimens after undergoing hemorrhagic shock following resuscitation using PEGylated Ox66™ particles in a Colloid, including complete survival of one specimen:

FIGS. 4A and 4B show systemic parameters including heart rate and mean arterial pressure following isovolemic hemodilution with test solutions;

FIG. 5 shows arteriolar luminal diameters. Arterioles included were smaller than 60 microns at baseline;

FIG. 6 shows the proliferation of hepatocarcinoma cells (HEPG-2) significantly reduced following administration with various concentrations of Ox66™;

FIG. 7A and FIG. 7B illustrate images of cells HEPG-2 cells prior to dosing and after dosing, respectively;

FIG. 8 shows the proliferation of prostrate carcinoma cells (22Rv1) significantly reduced following administration with various concentrations of Ox66™;

FIG. 9 shows the proliferation of lung carcinoma cells (A549) significantly reduced following administration with various concentrations of Ox66™;

FIG. 10 shows the proliferation of colon adenocarcinoma cells (CaCo-2) significantly reduced following administration with various concentrations of Ox66™;

FIG. 11 illustrates a bandage having a material impregnated with Ox66™; particles;

FIG. 12 illustrates a VAC system used in negative pressure wound therapy (NPWT), including a drape impregnated with Ox66™ particles;

FIG. 13 illustrates a scratch assay showing accelerated wound closure after dosing with Ox66™ particles, compared to a wound not dosed;

FIG. 14 illustrates a graph illustrated the wound closing over tame as shown in FIG. 13;

FIG. 15 illustrates a diaper having a material impregnated with Ox66™ particles; and

FIG. 16 illustrates a compound comprising Ox66™ and CBD.

### DESCRIPTION OF EXEMPLARY EMBODIMENTS

This disclosure is directed to a method of treating a mammal, including a human, comprising administering a therapeutically effective amount of a poly-oxygenated aluminum hydroxide composition to the mammal to improve mitochondrial function and efficiency, wherein the poly-oxygenated aluminum hydroxide composition comprises a clathrate containing bioavailable free oxygen gas (O₂) molecules. The poly-oxygenated aluminum hydroxide composition causes production of adenosine triphosphate (AT) in the mammal, including a human. The poly-oxygenated aluminum hydroxide composition comprises a clathrate containing bioavailable pure (100%) oxygen gas (O₂) molecules that are freely released to the mammal depending on the oxygen demand, i.e., more O₂ molecules released in hypoxic regions. The administration can be oral and the bioavailable O₂ molecules are time released into the mammal. The O₂ bioavailability of poly-oxygenated aluminum hydroxide composition can be slow if mammal, organ or tissue is well perfused and oxygenated, but rapid if hypoxic.

The following description of exemplary embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

An example of the poly-oxygenated aluminum hydroxide composition is Ox66™ manufactured by and available from Hemotek, LLC of Frisco, Tex. Ox66™ is a poly-oxygenated aluminum hydroxide having bioavailable oxygen gas (O₂) molecules and is composed of approximately 66.2% oxygen and organized as a true clathrate, allowing for the capture of oxygen molecules within its lattice structure. The disclosure avoids the applicational complications associated with conventional oxygen therapeutics, such as reliance on gaseous oxygen, systemic toxicity, and patient immobility. Ox66™ facilitates recovery of cells from injury while showing little to no significant toxicity.

Despite what is known from physiological principles, there is no practice-based evidence to suggest colloid solutions offer substantive advantages over crystalloid solutions with respect to hemodynamic effects. In addition, there is no evidence to recommend the use of other semisynthetic colloid solutions. Balanced salt solutions are reasonable initial resuscitation fluids, although there is limited practice-based evidence regarding their safety and efficacy. Additionally, the safety of hypertonic solutions has not been established. Ultimately, the selection of the specific resuscitative fluid should be based on indications, contraindications, and potential toxic effects in order to maximize efficacy and minimize toxicity. In addition, the capability of a resuscitative fluid to carry oxygen, as well as to maximize efficacy and minimize toxicity, is desperately needed.

There is a significant therapeutic benefit to intravenously oxygenate blood of a human individual and animal, collectively mammals, and create a more effective resuscitative fluid using a poly oxygenated metal hydroxide, and particularly nano-sized poly-oxygenated aluminum hydroxide, such as Ox66™ oxygen carrying particles. The Ox66™ poly-oxygenated aluminum hydroxide has a molecular formula Al₁₂H₄₂O₃₆ and the O_{2(g)} oxygen gas molecules are bioavailable to, and used by the body, because the O_{2(g)} oxygen gas molecules are not bound in the hydroxide complex. Ox66™ exists under STP (standard temperature and pressure) as a poly-oxygenated aluminum hydroxide comprising a clathrate, and chlorine. The molecular formula Al₁₂H₄₂O₃₆ is mathematically reduced to the molecular formula Al(OH)₃.6O₂. The 6 free oxygen gas molecules (O_{2(g)}) are separate from the oxygen molecules covalently bound in the hydroxide complex. The hydrogen is effervescent. The poly-oxygenated aluminum hydroxide is soluble in a fluid.

Ox66™ significantly increases tissue oxygenation of the mammal, known as oxygen tension PO₂. In certain applications of Ox66™, the PO₂ levels of a hemo-diluted mammal can exceed baseline. Fluid resuscitation with colloid and crystalloid solutions is a global intervention in acute medicine, and while the selection and ultimate use of resuscitation fluids is based on physiological principles, clinician preference determines clinical use. Studies have shown that Ox66™ does not create any negative effects in toxicology studies where Ox66™ was either injected or gavaged in a mammal.

With enough blood loss, like in amputations and other military trauma situations, red blood cell levels drop too low for adequate PO tissue oxygenation, even if volume expanders maintain circulatory volume they do not deliver oxygen. In these situations, the only currently available alternatives are blood transfusions, packed red blood cells, or a novel oxygen-enabled resuscitative fluid according to this disclosure.

This disclosure provides a novel oxygen-enabled blood additive, also referred to as a resuscitative fluid, that can effectively oxygenate mammal tissues and provide essential elements to protect and save critical cells and tissues, and the mammal itself. This disclosure is desperately needed on the battlefield, as well as in civilian trauma cases. One exemplary formulation consists of a fluid of 75-90% colloid or crystalline solutions with 10-25% addition of a poly-oxygenated metal hydroxide material, such as but not limited to, nano-sized Ox66™ particles, resulting in concertation ranges 0.1 mg/l to 1000 mg/l. For use as a blood additive, ideal sizes of the Ox66™ particles may be between 10 nm to 100 μm in size, depending on the treatment. To avoid immune response, it is critical in some treatments that the diameter of the Ox66™ particles should ideally be less than 300 nm as these particle sizes have less potential for toxicity and maximized efficacy,

The blood additive compositions can include surface modifications of nano-sized poly-oxygenated metal hydroxide particles with polyethylene glycol (PEG) for increased vascular transit, protein for increased surface to volume ration, or specific charge to enhance absorption and sustained PO₂. These modifications of the poly-oxygenated metal hydroxide material as a blood additive extend the oxygenating capabilities of the material for longer periods of time, thus extending product life, such as specifically in far-forward combat theatres.

This blood additive composition is extremely significant because the blood additive is agnostic to the blood type of a mammal, meaning that the blood additive can be administered to a human individual without typing the human individual's blood. Thus, even individuals with rare blood types can be effectively treated with the same blood additive. There is no time delay as the blood additive can be immediately administered to an individual in a crisis situation. Further, the blood additive has significant shelf life and can be stored at room temperature in locations where administration of the blood additive can be performed in emergency situations, such as in the battlefield to extend a soldier's life until the soldier can be transported to a quality hospital, or in an ambulance or fire truck. Stabilizing a human individual for hours or even minutes can save a human individual's life.

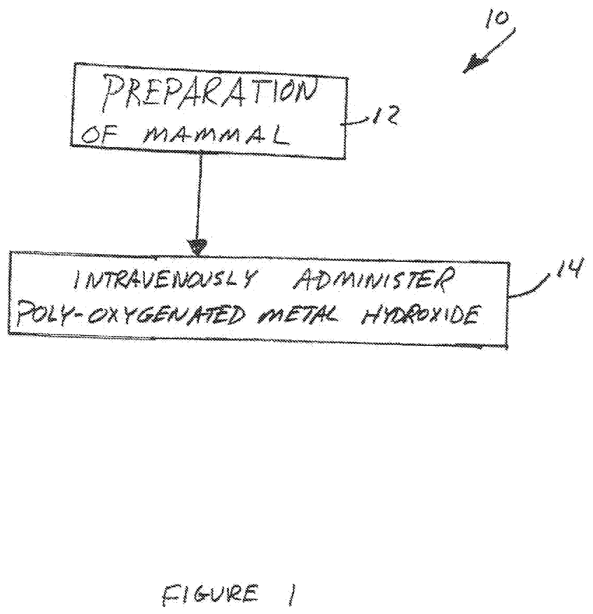

As shown in FIG. 1, this exemplary embodiment comprises a method 10 of intravenously administering a mammal a therapeutic amount of a composition including a poly-oxygenated metal hydroxide, such as a human individual, or an animal. The poly-oxygenated metal hydroxide composition may comprise a poly-oxygenated aluminum hydroxide, such as Ox66™ particles. One method includes administration of a therapeutically effective resuscitative fluid to increase tissue oxygenation PO₂ in the mammal. Another method can include administration of a therapeutically effective composition to treat a condition of a mammal. The method comprises preparing a mammal at step 12, such as preparing a site on the mammal for receiving a catheter, and intravenously administering a poly-oxygenated metal hydroxide composition at step 14, such as using a catheter. Various methods and treatments are detailed as follows.

### Study

A preclinical study was performed m ascertain the efficacy of a poly oxygenated metal hydroxide in a mammal, comprising Ox661™ particles, and the details of the study and results are included. For this study, Particle Size A diameter is 100 um and Particle Size B diameter is 10 um.

In this study, male Sprague-Dawley rats underwent a 50% blood volume isovolemic hemodilution exchange with either Ox66™ or phosphate buffered saline (PBS; volume control), since Ox66™ was suspended in PBS, such as lactated Ringers solution (LRS). LRS is a crystalloid electrolyte sterile solution of specified amounts of calcium chloride, potassium chloride, sodium chloride, and sodium lactate in water for injection. LRS typically is used intravenously to replace electrolytes. Isovolemic hemodilution is the reduction of red blood cells (hematocrit) with an equal volume of hemodiluent, i.e., crystalloids, colloids or oxygen therapeutics.

The withdrawal/infusion rate was 2.0 ml×min⁻¹×kg⁻¹ and performed through a cannulated carotid artery and jugular vein. Systemic measurements were recorded via a cannulated femoral artery that was connected to a pressure transducer (MP150; Biopac Systems, Inc. Goleta, Calif.), while microcirculatory parameters were collected through phosphorescence quenching and intravital microscopic examination of the exteriorized spinotrapezius muscle. Compared to baseline, a 50% blood volume exchange with either hemodiluent caused a reduction in heart rate, blood pressure arterial diameter and interstitial fluid (ISF) oxygen tension (PO₂) in all animals. However, Ox66™ animals demonstrated an improvement in ISF PO₂ compared to PBS animals. This finding demonstrates that Ox66™ both transports and releases oxygen to the peripheral microcirculation.

Animals Male Sprague Dawley rats (250-300 g) Anesthetics Isoflurane (induction) Alfaxalone (continuous rate of infusion)

Surgical Preparation. Vessels and tracheal cannulation Spinotrapezius muscle exteriorized

Systemic Parameters BIOPAC MP150 (real-time analysis)

Tissue Oxygenation Phosphorescence Quenching Microscopy Palladium porphyrin (R0) probe distributed into interstitium. Phosphorescence decay curve captured and lit to standard curve for translation. to NSF O2 in mmHg.

Hemodilution (HD) Baseline parameters collected 50% isovolemic exchange of blood with test solution at 2.0 ml/kg/min Post-HD parameters collected Animals observed for 2 h post-HD

Hemodiluents Phosphate Buffered Saline (PBS) Ox66™ Size A[1×] Ox66™ Size A[10×] Ox66™ Size B[10×] Ox66™ Size B[10×]

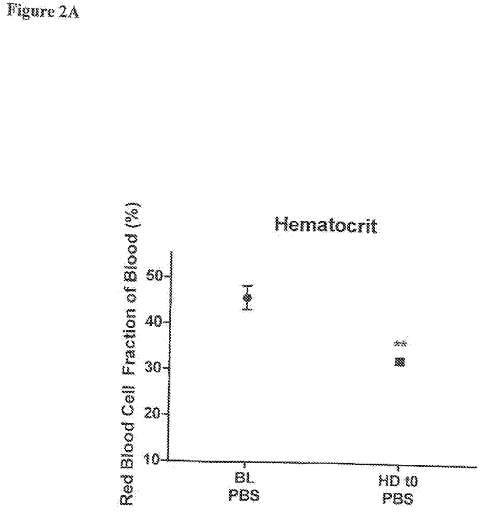

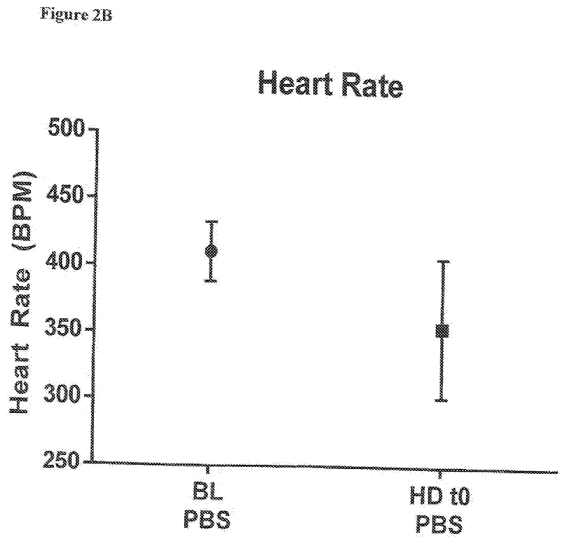

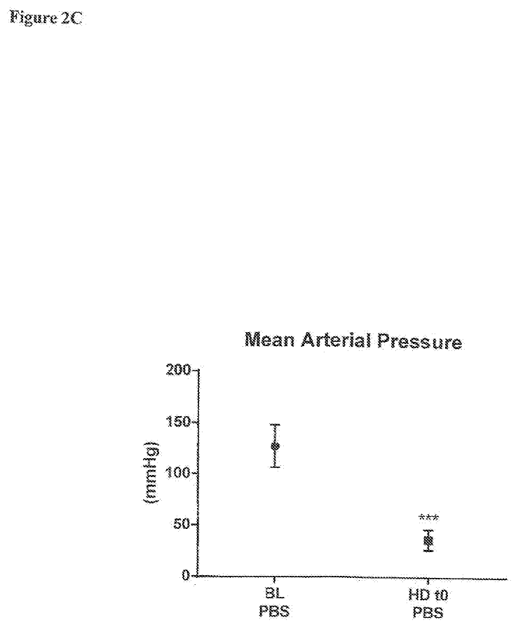

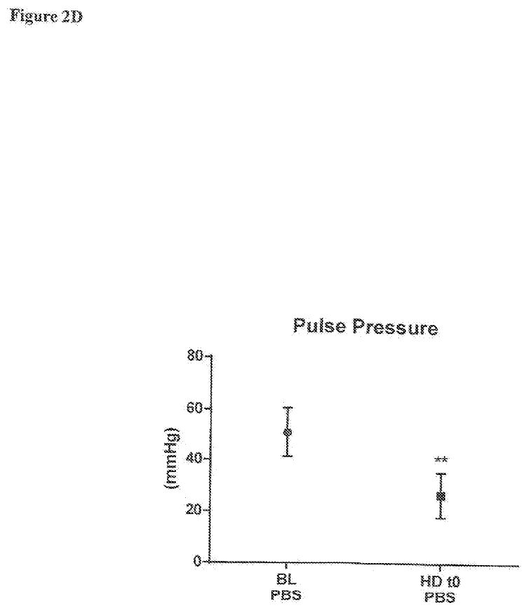

FIGS. 2A-2D show systemic characteristics of 50% isovolemic hemodilution (HD). Measurements were taken immediately prior to baseline (BL) and following hemodilution at (HD t0). The volume exchange of whole blood with PBS (vehicle volume control) resulted in significant reductions in hematocrit, mean arterial pressure, and pulse pressure. The reduction in heart rate lacked statistical strength. **p<0.01, ***p<0.001.

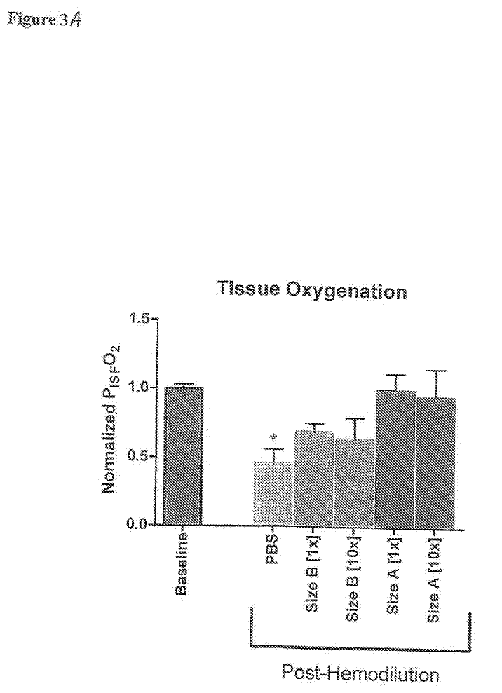

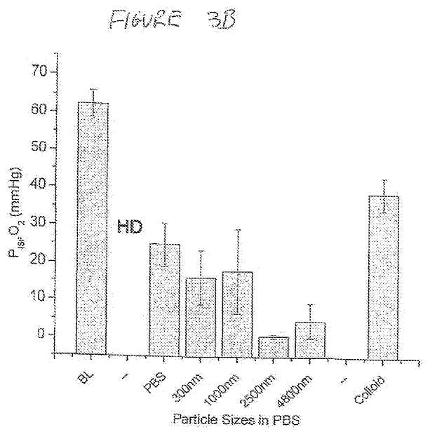

FIG. 3A shows tissue oxygenation (P_{ISF}O₂) following 50% volume replacement. All P_{ISF}O₂ values (mmHg) were normalized to baseline (BL) for ease of comparison. PBS alone was used as a vehicle volume control. Ox66™ compounds were suspended in PBS as crystalloids, where particle size A was 10× larger than particle size B and trended towards higher oxygen delivery, Both particle sizes were assessed at 1× and 10× concentrations, but failed to show a concentration dependence of P_{ISF}O₂ in this range. *p<0.05 vs BL. Particle Size A diameter is 100 um and Particle Size B diameter is 10 um.

FIG. 3B shows tissue oxygenation (P_{ISF} O₂) following 50% volume replacement. All P_{ISF} O₂ values (mmHg) were normafized to baseline (BL) for ease of comparison. PBS alone was used as a vehicle volume control. FIG. 3B shows Ox66™ particles diameters being smaller than those shown in FIG. 3A that were suspended in PBS as crystalloids, having sizes of 300 nm, 1000nm (1 um), 2500 nm (2.5 um), and 4800 nm (4.8 um), compared to the PBS alone. Compared to the results shown in FIG. 3A, Ox66™ particles having a diameter of around 10 um suspended in PBS as a crystalloid appear to achieve a superior increase in P_{ISF} O₂ values (mmHg).

FIG. 3B also shows Ox66™ particles suspended in a Colloid that results in vastly improved P_{ISF} O₂ values (mmHg) compared to PBS alone, and also compared PBS including Ox66™ particles as a crystalloid having reduced size particles, as shown. This is due in part to the blood additive composition including surface modifications of the nano-sized poly-oxygenated metal hydroxide particles with polyethylene glycol (PEG) for increased vascular transit, protein for increased surface to volume ration, and/or specific charge to enhance absorption and sustained PO₂. The PEGylation particles have a spherical shape that makes them more slippery which results in better capillary transit and less irritation of the capillaries. The PEGylation also serves as an aggregate inhibitor. These modifications of the poly-oxygenated metal hydroxide material as a blood additive provides increased concentration control and extends the oxygenating capabilities of the material for longer periods of time, thus extending product life, such as specifically in far-forward combat theatres,

FIG. 3C shows the results of resuscitation of five male Sprague-Dawley rat specimens after hemorrhagic shock. As shown, two specimens underwent resuscitation with a Colloid including 2.4 um Ox66™ PEGylation particles, and each specimen survived 1 hour after hemorrhagic shock. This is significant as death would have occurred within 10 minutes of hemorrhagic shock.

Even more significant one of the three specimens that underwent resuscitation with a Colloid including 4.8 um Ox66™ PEGylation particles showed a significant immediate increase in P_{ISF}O₂, and survived 8 hours after hemorrhagic shock, when the monitoring was completed and the specimen continued to survive, a complete survival. A second specimen showed a significant immediate increase in P_{ISF}O₂ and survived 3 hours. The third specimen also survived an additional 3 hours. This significant survival of all five specimens after hemorrhagic shock by resuscitating each with a Colloid including Ox66™ PEGylation particles is remarkable. Advantageously, survival from hemorrhagic shock without using a blood product is extremely encouraging, as the Colloid does not require blood typing. When used on individuals on the battlefield, this survival time is significant and allows transport of an individual that undergoes hemorrhagic shock to a treatment facility.

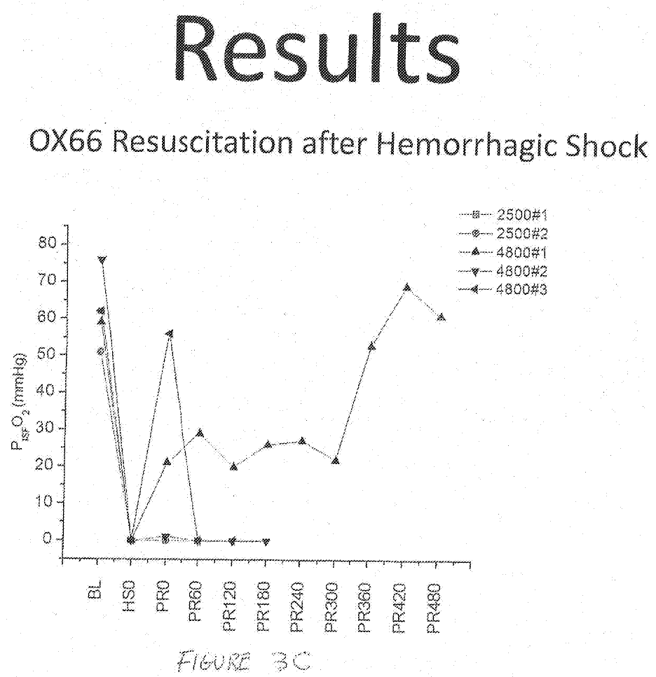

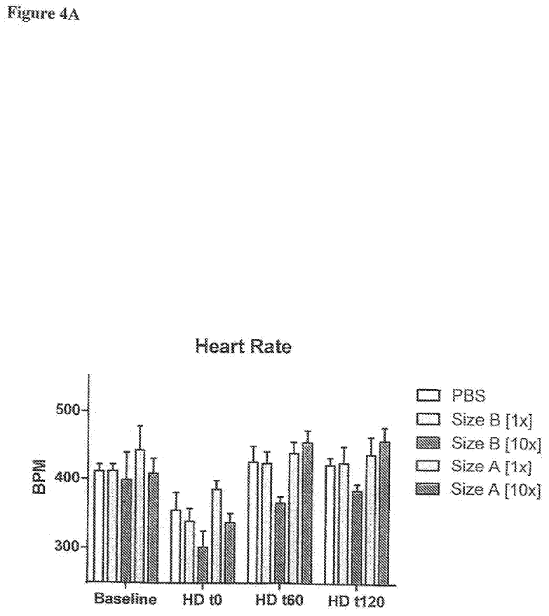

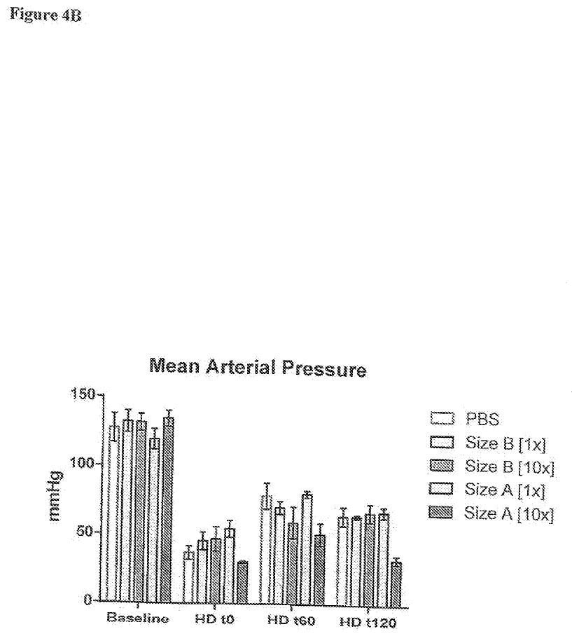

FIGS. 4A and 4B shows systemic parameters following isovolemic hemodilution with test solutions. HD=Hemodilution; in time point in minutes following hemodilution shown in FIG. 4A, heart rates generally followed the scheme of slowing down by HD t0 and then returning to baseline by t60. As shown FIG. 4B, mean arterial pressure remained low, but stable following hemodilution with the exception of Size A at 10× concentration. Statistical tests were not performed due to low sample sizes (N=2-4).

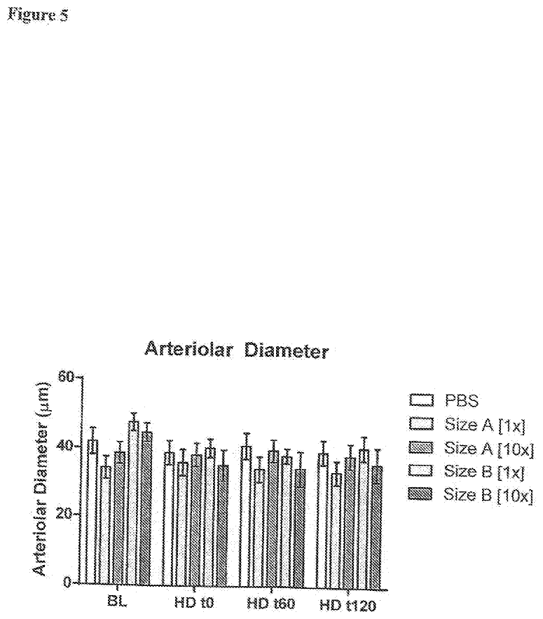

FIG. 5 shows arteriolar luminal diameters. Arterioles included were smaller than 60 microns at baseline.

### SUMMARY

The ‘50% Isovolemic Hemodiltuion’ model produces a good reduction in systemic cardiovascular parameters and tissue oxygenation to assess therapeutic potential of interventions.

Ox66™ is capable of carrying and delivering oxygen to hypoxic peripheral tissues.

### Administering Surface Modified Ox66™ Particles

In an exemplary embodiment, the administered Ox66™ particles may be surface modified for specific therapeutic uses such as time release, PEGylation, growth factor modification, antibacterial, antimicrobial, protein modification, and enzymes.

### Treatment of Traumatic Brain Injury (TB Strokes, and CTE

To achieve microcirculation in mammals, such as to treat TBI and strokes, the Ox66™ particles preferably have a diameter of less than 300 nm to pass the blood brain barrier (BBB). The upper limit of pore size enabling passive flow across the BBB is usually <300 nm; however, particles having a diameter of several nanometers can also cross the BBB via carrier-mediated transport using specialized transport proteins. Alternatively, receptor-mediated transport can act as an “escort” for larger particles. This exemplary embodiment comprising in administering a therapeutic amount of a composition including Ox66™ particles having a diameter of less than 300 nm is therapeutically effective in treating a mammal with TBI and BBB. This is an extraordinary accomplishment, and can revolutionize the treatment of not only TBI and BBB, but also other brain conditions/injury including Chronic Traumatic Encephalopathy (CTE), which is a progressive degenerative disease of the brain found in athletes, military veterans, and others with a history repetitive brain trauma.

### Treatment of Diabetes

To achieve microcirculation in mammals to treat Diabetes, this exemplary embodiment comprises intravenously administering to a mammal a therapeutic amount of a composition including Ox66™ particles as a fluid that is therapeutically effective to increase PO₂ in the mammal, such as a human individual, or an animal, to reduce the effects of Diabetes.

### Treatment of Carcinoma

To treat cancer in mammals, exemplary embodiments comprise intravenously administering to a mammal a therapeutic amount of a composition including Ox66™ particles as a fluid that is therapeutically effective to reduce the effects of, or eliminate, cancer cells in the mammal, such as a human individual, or an animal. The composition Ox66™ can also be administered orally to the mammal.

The charts in the Figures described hereafter illustrate laboratory results of the proliferation of the identified carcinoma after administration of various concentrations of the Ox66™ in a fluid to living carcinoma cells compared to control, which is no administration of the OX66™ to the cells.

For the 161lowing results, three assays are used: Janus Green (JG) colorimetric assay, Lactase Dehydrogenase (LDH) colorimetric assay, and CFDA-5 fluorometric assay.

Janus Green (JG) is a supravital stain, meaning it is absorbed by damaged cells. It is not able to penetrate healthy cells, but when cells are damaged or dead, it is able to pass easily into the cell, and stain the mitochondria. Janus Green is a relatively quick way to assess the heath of cells, and it must be used in two parts; one plate for viability, and the other for proliferation in order to obtain a percent viability of cells. The measurements are not exact numbers, but rather an estimate based on professional observation.

### Janus Green Protocol:

Obtain two (2) 96-well plate (one plate for viability, the other plate for proliferation). Seed ˜1 Million identified living carcinoma cells per plate,

Once the carcinoma cells have reached 50% confluency (˜24 hours), dose the cells in the plates with varying concentrations of Ox66™ fluid (2 columns of cells for each concentration of Ox66™ tiding control).

After 24 hours, run JG.

Standard Protocol was followed:

For the viability, the cells were stained with JG dye before being fixed with 100% ethanol. This shows which cells were still alive.

For the proliferation, the cells were fixed with 100% ethanol of before being stained with JG to get an approximate number of how many cells were seeded.

The plates were then run in a colorimetric plate reader.

Lactate dehydrogenase is an enzyme that is present in all li cells, and is released when cell membrane integrity is compromised, making this assay, which detects the presence of LDH a reliable option for cytotoxicity. The LDH assay uses the compound iodonitrotetrazolium (INT) to react with LDH present to form a red colored formazan. This react can then be read under a colorimetric plate reader and be quantified.

### LDH Protocol:

Seed and dose the carcinoma cells the same as for JG, with only one 96-well plate.

50 microliters of cell media are taken from each well and placed into a new well plate, then 50 microliters of LDH solution is added to the new well plate, along with the media.

The plate was then run in a colorimetric plate reader.

5-CFDA, AM assay is an enzymatic marker assay, as well as a cell membrane permeability marker. Enzymatic activity present within the cells will cause the CFDA dye to fluoresce, and the cell membrane integrity will retain the fluoresced product within the cell.

5-CFDA, AM Protocol:

Seed and dose the cells the same as for LDH.

The cells are stained with the CFDA dye and are incubated for ˜30 minutes, then the solution is diluted with media, and read under a fluorescent plate reader.

### STUDY 1-Liver Carcinoma (HEPG-2)

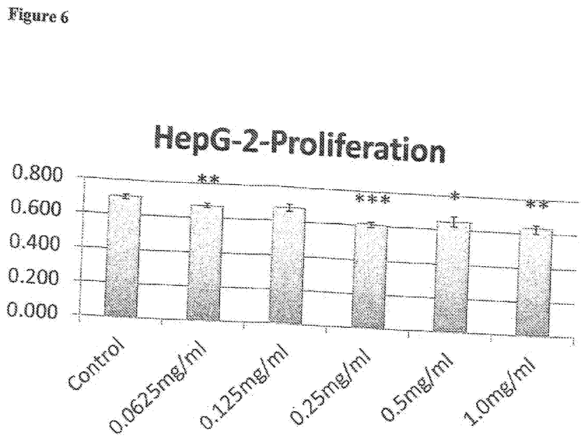

The proliferation of hepatocarcinoma cells (HEPG-2) was significantly reduced following administration of various concentrations of Ox66™ to the cells, as shown in FIG. 6. A hypoxic microenvironment, which is a common feature of hepatocellular carcinoma can induce HIF-1α expression and promote the epithelial-mesenchymal transition (EMT). Additionally, it can induce the invasion of cancer cells. This proven characteristic of hepatocarcinoma supports the hypothesis that Ox66™ is effective in reducing the proliferation of these cells.

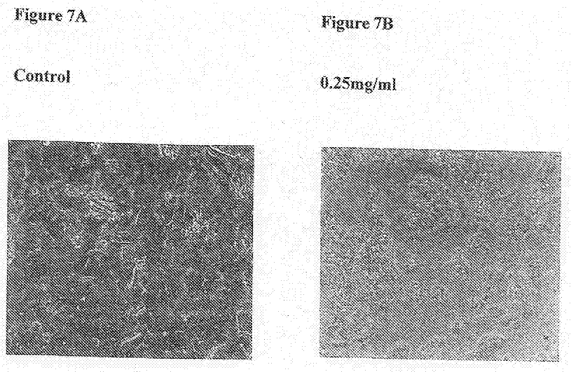

Images shown in FIG. 7A and FIG. 7B illustrate HEPG-2 cells prior to dosing and after dosing with Ox66™ fluid, respectively.

### STUDY 2-Prostate Carcinoma (22Rv1)

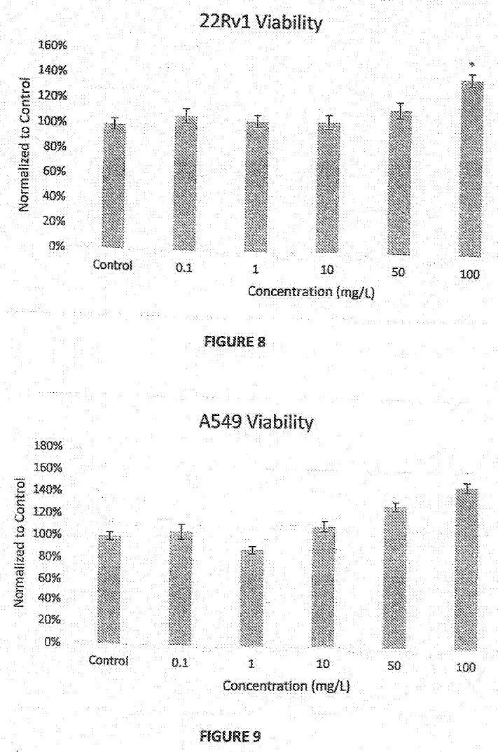

The proliferation of prostrate carcinoma (22Rv1) cells was significantly reduced following administration with various concentrations of Ox66™ fluid to the cells, as shown in FIG. 8. Prostrate carcinoma cells are hypoxic, which helps explain why Ox66™ is effective in reducing the proliferation of these cells.

For this cell line, 22Rv1, the Janus Green colorimetric assay was used to determine how viable the cell is were after being dosed with varying concentrations of the Ox66™ into the cell culture media. This administration is similar to injection into the blood stream as would be given via an intravenous injection (IV). Janus Green is an exclusion dye, which only stains mitochondria and nuclei of damaged cells. For the assay, the cell culture was washed twice with phosphate buffered saline (PBS), followed by one minute fixation with absolute ethanol. The culture was then subjected to one-minute staining by Janus Green B dye solution followed by two PBS wash to remove the excess dye. Then the encapsulated dye from these cells was extracted with absolute ethanol, and an additional 100 ul water was added to each well to maintain samples. Optical intensity was then read at 630 nm on a microplate reader. Janus Green gives intensive staining of the nuclei with light staining of the cytoplasm, thus outlining cells clearly. Therefore, morphologic changes of cells can also be screened after the assay using an inverted microscope. The more Janus Green present, the more damaged or dead cells are present as well. The graph shows that for administration of Ox66™ fluid to the cells at a concentration of 100 mg/L, there is a statistical difference between the uptake of Janus Green at 100 mg/L than at 0 mg/L, or the control. This is the only concentration that is statistically different when compared to the control for this carcinoma.

STUDY 3-Lung Carcinoma (A549)

The proliferation of lung carcinoma (A549) cells was significantly reduced following administration with various concentrations of Ox66™ fluid to the cells, as shown in FIG. 9. Lung carcinoma cells are hypoxic, which helps explain why Ox66™ is effective in reducing the proliferation of these cells.

For this cell line, A549 (lung carcinoma), the Janus Green colorimetric assay was used to determine how viable the cells were after being dosed with varying concentrations of Ox66™ into the cell culture media. This administration is similar to injection into the blood stream as would be given via an intravenous injection (IV). Janus Green is an exclusion dye, which only stains mitochondria and nuclei of damaged cells. For the assay, the cell culture was washed twice with phosphate buffered saline (PBS), followed by one minute fixation with absolute ethanol. The culture was then subjected to one-minute staining by Janus Green B dye solution followed by two PBS wash to remove the excess dye. Then the encapsulated dye from these cells was extracted with absolute ethanol, and an additional 100 ul water was added to each well to maintain samples. Optical intensity was then read at 630 nm on a microplate reader. Janus Green gives intensive staining of the nuclei with light staining of the cytoplasm, thus outlining cells clearly. Therefore, morphologic changes of cells can also be screened after the assay using an inverted microscope. The more Janus Green present, the more damaged or dead cells are present as well. The graph shows that for the administration of Ox66™ at 50 mg/L and 100 mg/L there is a statistical difference between the uptake of Janus Green at 50 mg/L and 100 mg/L than at 0 mg/L, or the control. This indicates that these carcinoma cells are more receptive to the Ox66™ treatment than 22Rv1 cells.

### STUDY 4-Colon Adenocarcinoma (CaCo-2)

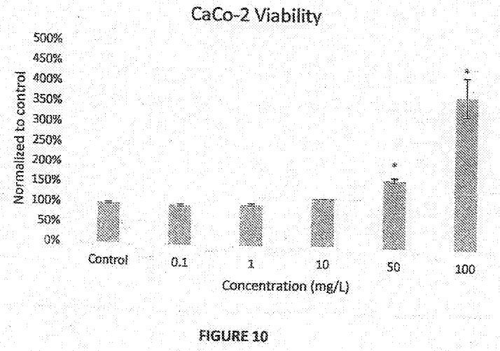

The proliferation of colon adenocarcinoma cells (CaCo-2) was significantly reduced following administration with various concentrations of Ox66™ in the culture media of the cells, as shown in FIG. 10. Colon adenocarcinoma cells are hypoxic, which helps explain why Ox66™ is effective in reducing the proliferation of these cells.

For this cell line, CaCo-2 (colon adenocarcinoma), the Janus Green colorimetric assay was used to determine how viable the cells were after being dosed with varying concentrations of Ox66™ into the cell culture media. This administration is similar to injection into the blood stream as would be given via an intravenous injection (IV) fluid, Janus Green is an exclusion dye, which only stains mitochondria and nuclei of damaged cells. For the assay, the cell culture was washed twice with phosphate buffered saline (PBS), followed by one minute fixation with absolute ethanol. The culture was then subjected to one-minute staining, by Janus Green B dye solution followed by two PBS wash to remove the excess dye. Then the encapsulated dye from these cells was extracted with absolute ethanol, and an additional 100 ul water was added to each well to maintain samples. Optical intensity was then read at 630 nm on a microplate reader. Janus Green gives intensive staining of the nuclei with light staining of the cytoplasm, thus outlining cells clearly. Therefore, morphologic changes of cells can also be screened after the assay using an inverted microscope. The more Janus Green present, the more damaged or dead cells are present as well. The graph shows that for administration of Ox66™ at 50 mg/L and 100 mg/L there is a statistical difference between the uptake of Janus Green at 50 mg/L and 100 mg/L than at 0 mg/L, or the control. This indicates that these cells tare more receptive to Ox66™ than 22Rv1 cells, There is a substantial jump in uptake of the Janus Green at 100 mg/L, meaning there were many more damaged cells at this concentration.

### Erectile Dysfunction

To achieve the treatment of erectile dysfunction in mammals, this exemplary embodiment comprises intravenously administering to a mammal a therapeutic amount of a composition including Ox66≮ particles that is therapeutically effective to increase oxygenated blood flow thus mitigating physical dysfunction in the mammal, such as a human individual, or an animal, to reduce the effects of erectile dysfunction. In another embodiment, the Ox66™ particles could be embodied in a tablet or capsule form and administered orally.

### Sickle Cell Anemia

To achieve the treatment of sickle cell anemia as mammals, this exemplary embodiment comprises intravenously administering to a mammal a therapeutic amount of a composition including Ox66™ particles that is therapeutically effective to increase oxygenated blood flow thus mitigating dysfunction in the mammal, such as a human individual, or an animal, to reduce the effects of sickle cell anemia. In another embodiment, the Ox66™ particles could be embodied in a tablet or capsule form and administered orally. In sickle cell anemia, the red blood cells become rigid and tacky and are shaped like sickles hence the name of the disease. These irregularly shaped “sickle” cells do not move through small blood vessels, resulting in slowing or blockage of blood flow and oxygen to parts of the body. This embodiment of Ox66™ particles could oxygenate the body in a crisis and act as an alleviation strategy for sickle cell anemia.

### Bronchopulmonary Dysplasia (BPD)

To treat bronchopulmonary dysplasia in mammals this exemplary embodiment comprises intravenously administering to a mammal a therapeutic amount of a composition including Ox66™ panicles as a fluid that is therapeutically effective to reduce the elects of, or) eliminate, BPD in the mammal, such as a human individual, or an animal. A critical problem facing preterm infants is adequate lung function. Premature babies can have strokes, chronic lung disease and potential brain damage due to small, fragile blood vessels, and pressurized oxygen required after birth to keep the lungs functional. There is a need for an alternative oxygen therapy that mitigates the aforementioned risks. These preemies frequently encounter complications such as chronic lung disease—sometimes called bronchopulmonary dysplasia (BPD). BPD can occur because the infants still have sonic inflammation in their lungs and may require extra oxygen or medications to help them breathe comfortably. There are several hyper-oxygenated associated illnesses that a preterm infant will suffer such as retinopathy of prematurity (ROP), periventricular leukomalacia, cerebral palsy, and the previously mentioned bronchopulmonary dysplasia (BPD), to name a few. Administration of Ox66™ provides alternative oxygen delivered by less invasive means yet supplying oxygen to the preterm infant.

### Alzheimer's Disease (AD)

To treat Alzheimer's disease in mammals, this exemplary embodiment compasses intravenously administering to a mammal a therapeutic amount of a composition including Ox66™ particles as a fluid that is therapeutically effective to reduce the effects of or eliminate, AD in the mammal, such as a human individual, or an animal. Alzheimer's disease (AD) is classified as a neurodegenerative disorder. The cause and progression of the disease are not well understood. AD is associated with hallmarks of plaques and tangles in the brain. Current treatments only help with the symptoms of the disease and there are no available treatments that stop or reverse the progression of the disease. As of 2012, more than 1,000 clinical trials have been or are being conducted to test various compounds in AD. There is currently no approved drug therapy for AD that will stop or reverse the progression of the disease. There is a clear link between low oxygen levels in the brain and Alzheimers disease, but the exact mechanisms behind this are not yet fully understood (Alzheimer's Society, Proceedings of the National Academy of Sciences). A healthy brain needs a good supply of oxygen. A disruption of the blood flow through or to the brain causes low oxygen levels. When there is damage or a blockage, or the blood supply itself is low in oxygen then insufficient oxygen will be delivered to the brain cells. Ox66™ offers the potential of micrometer sized (˜0.07 μm) particles increasing oxygen delivery to the brain. With this offloading of oxygen, there is significant potential to mitigate the development and/or the progression of Al.:

### Autism

To treat autism in mammals, this exemplary embodiment comprises intravenously administering to a mammal a therapeutic amount of a composition including Ox66™ particles as a fluid that is therapeutically effective to reduce the effects of, or eliminate, autism in the mammal, such as a human individual, or an animal. Several problems that crop up during labor and shortly after birth appear to increase a child's risk for developing, autism. A recent study published in the journal of Pediatrics, a review of 40 studies published before April 2007, looked at a host of circumstances that may affect babies during labor and delivery. It found 16 circumstances that appear to be tied to a significantly increased risk that a child would develop autism later in life. Researchers note that many of these complications tend to occur together in difficult or high-risk deliveries, making it difficult to finger a single suspect. But broadly, researchers note, they seem to be related to oxygen deprivation and growth retardation.

### Wound Care

This portion of the disclosure is directed to wound care using a material impregnated with Ox66™ particles, such as bandage-type dressings, and vacuum-assisted closure (VAC) system, to provide efficient oxygen delivery to injured tissue. The impregnated material avoids the applicational complications associated with conventional oxygen therapeutics, such as reliance on gaseous oxygen, systemic toxicity, and patient immobility.

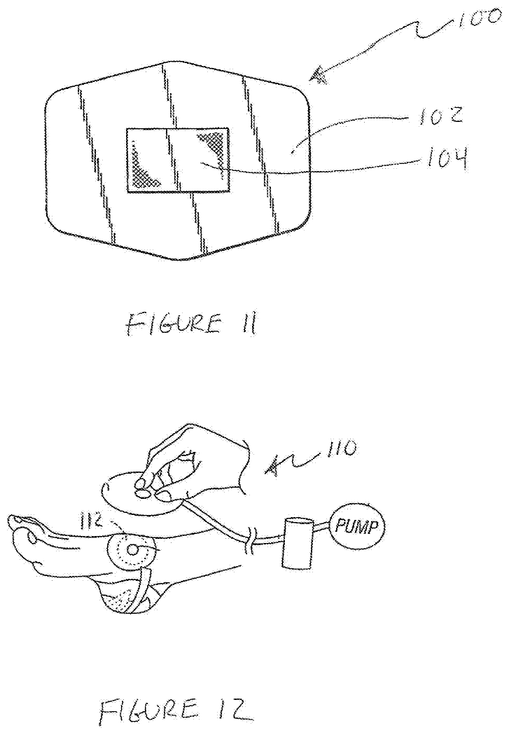

Referring to FIG. 11, there is shown an example of a bandage-type dressing at 100, such as a self-adhering bandage comprising a carrier strip 102 having an adhesive layer disposed thereon, and a centrally located fluid absorbing material strip material 104, such as gauze, impregnated with Ox66™ particles. Impregnated in this disclosure is defined as to be filled, imbued, permeated, or saturated, to permeate thoroughly. The file dressing, including the Ox66™ particles, is sterile. The impregnated material can be selected from various types of fluid absorbing materials and limitation to gauze is not to be inferred. The dressing can also comprise a non-adhesive based dressing, such as a roll of gauze.

Advantages of the impregnated material is that the Ox66™ particles are a fine powder and will remain in contact with and proximate to a wound at a specific location for an extended time. Moreover, the amount of the Ox66™ particles per unit area can be precisely defined, which is beneficial to effect desired treatment of a wound, and to remove waste of unused powder. The Ox66™ particles are particularly effective for treating wounds of various types a will be described shortly.

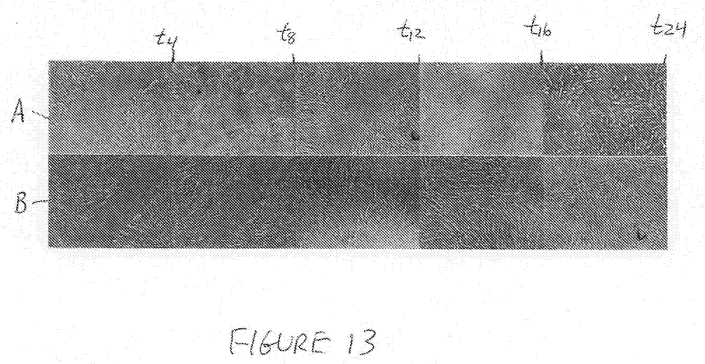

FIG. 12 shows a VAC system 110 used in negative pressure wound therapy (NPWT), which is used for various compromised dermal conditions. A sterile drape 112 is shown that is impregnated with Ox66™ particles in another example of this disclosure.

A scratch assay is a well-developed, in vitro alternative for studying cell migration. One of the foremost advantages of this method is that it mimics the migration of cells in vivo where an incisional wound might be studied. The scratch assay functions as an in vitro alternative to a physical injury.

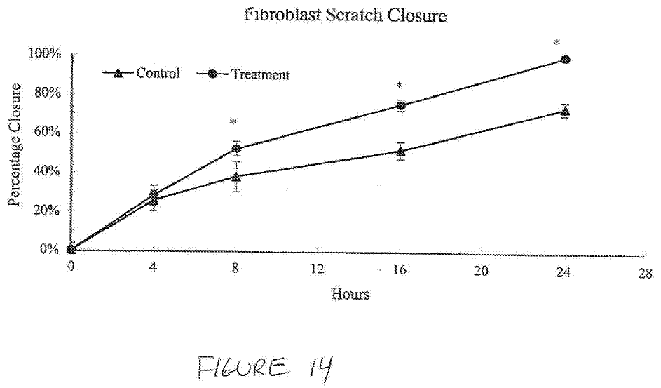

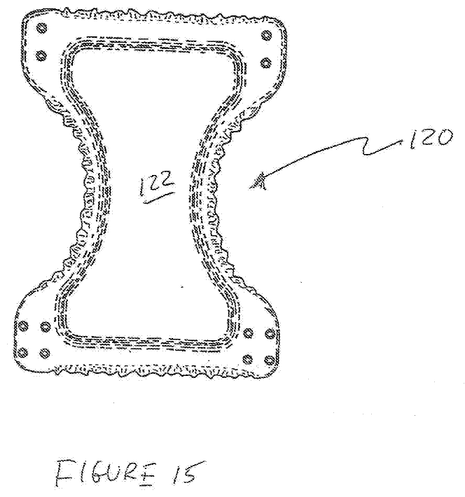

As hewn in FIG. 13, there is shown a scratch assay in treatment groups such as shown at A, where the percentage closure of the scratch dosed with Ox66™ in comparison to its initial width roughly increased at similar rates after each time point with 28% after 4 h, 24% after 8 h. 17% after 16 h and 25% after 24 h, as graphically shown in FIG. 14. Based on the data observed, cells migrated at an approximately constant rate showing linear closure at each measured time point. Contrarily, as graphically shown in FIG. 14, cells in the control groups as shown at B not dosed with Ox66™ started at a higher rate of migration for the first 4 h with a 26% mean closure than the subsequent time points. Migration rate slowed down to between 12 and 14% of mean closure from 4 h post-injury to 16 h post-injury. During, the final observation period, the mean closure rate resumed to 21%, and concluded in an overall 73% mean closure at the end of experiments. In both sets A and B, as time progressed, the buildup of cellular debris became more evident. This is believed to be due to the sloughing of dead cells during migration and regeneration in the wound healing process.

### Diapers

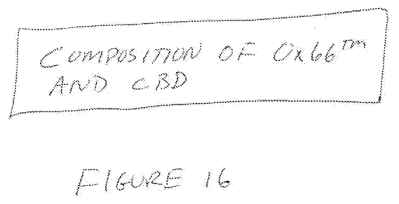

Referring now to FIG. 15, there is shown a diaper 120 comprising an injury absorbing material 122 impregnated with Ox66™ particles. The Ox66™ particles help reduce urine and other insults from creating diaper rash on a patient, such as to a baby's or an adult's skin. In addition, the Ox66™ particles help to neutralize some of the ammonia in urine. The Ox66™ particles also dissolve in the urine as Ox66™ particles are soluble up to 1g/L. Thus, the Ox66™ particles are moisture activated when comprised in the diaper.

### Ox66™ Particles and CBD

According to this disclosure, a composition comprising Ox66™ particles and CBD, as shown in FIG. 16, provides health benefits to mammals. The unique blend of Ox66™ particles providing free oxygen gas, a manufactured material, and CBD, a naturally occurring substance, provides a composition that treats numerous benefits to a mammal,

Ox66™ particles provide numerous benefits, including use on skin care, and wound care as previously discussed.

Cannabidiol (CBD) has some health benefits, including therapeutic benefits. CBD is one of many compounds, known as cannabinoids, in the cannabis plant. CBD oils are oils that contain concentrations of CBD. The concentrations and the uses of these oils vary.

Until recently, the best-known compound in cannabis was delta-9 tetrahydrocannabinol (THC). This is the most active ingredient in marijuana. Marijuana contains both THC and CBD, and these compounds have different effects.

THC creates a mind-altering “high” when a person smokes it or uses it in cooking. This is because THC breaks down when heat is applied and then introduced into the body.

CBD is different. Unlike THC, it is not psychoactive. This means that CBD does not change a person's state of mind when they use it. However, CBD does appear to produce significant changes in the body, and some research suggests that it has medical benefits.

The least processed form of the cannabis plant is hemp. Hemp contains most of the CBD that people use medicinally. Hemp and marijuana come from the same plant, Cannabis sativa, but the two are very different. Over the years, marijuana farmers have selectively bred their plants to contain high levels of THC and other compounds that interested them, often because the compounds produced a smell or had another effect on the plant's flowers. However, hemp farmers have rarely modified the plant. These hemp plants are used to create CBD oil.

All cannabinoids, including CBD, produce effects in the body by attaching to certain receptors. The human body produces certain cannabinoids on its own. It also has two receptors for cannabinoids called the CB1 receptors and CB2 receptors.

CB1 receptors are present throughout the body, but many are in the brain. The CB1 receptors in the brain deal with coordination and movement, pain, emotions, and mood, thinking, appetite, and memories, and other functions. THC attaches to these receptors.

CB2 receptors are more common in the immune system. They affect inflammation and pain. Researchers once believed that CBD attached to these CB2 receptors, but it now appears that CBD does not attach directly to either receptor. Instead, it seems to direct the body to use more of its own cannabinoids.

CBD may benefit a person's health in a variety of ways. For example, CBD can be used as a natural pain relief and anti-inflammatory, and for treating chronic pain. People tend to use prescription or over-the-counter drugs to relieve stiffness and pain, including chronic pain. Some people believe that CBD offers a more natural alternative. CBD significantly reduces chronic inflammation and pain in some mice and rats.

Acne treatment is another promising use for CBD. The condition is caused, in part, by inflammation and overworked sebaceous glands in the body. CBD also helps to lower the production of sebum that leads to acne, partly because of its anti-inflammatory effect on the body. Sebum is an oily substance, and overproduction can cause acne. CBD could become a future treatment for acne vulgaris, the most common form of acne.

### Mitochondrial Dysfunction

Sarcopenia is defined as an age related, involuntary loss of skeletal muscle mass, strength and function that can lead to frailty syndrome. Although sarcopenia is a disease of the elderly, it has been associated with conditions not related to aging, and strongly linked to hypoxia. It is well documented that exposing humans to a hypoxic environment, especially above 5,000 meters, a hypoxic induced sarcopenia with rapid loss of muscle mass occurs. Studies of climbers in their attempt to conquer Mount Everest lose a significant of muscle mass independent of other causes such as nutritional deficiencies. In addition, sarcopenia of aging is strongly linked to hypoxic disease processes of aging. Comorbidities such as chronic obstructive pulmonary disease (COPD), hypoxia, and peripheral artery disease strictly correlate with development and accelerated onset of sarcopenia with physical disability, poor quality of life, and significant morbidity and mortality. Sarcopenia poses a major burden and cost on the global health care system.

Diseases related to hypoxia start to develop in middle age and increase in incidence in the later decades of life. Patients with heart failure, COPD and peripheral artery disease (PAD) typically experience muscle wasting, that is 10 to 40% greater in magnitude than healthy matched patients of similar age. The hypoxic induced sarcopenia patients experience significant reduced strength and physical function. Hypoxia induces a loss of mammalian target of rapamycin (mTOR), a muscle growth stimulating signaling protein, and suppresses messenger ribonucleic acid (mRNA) translation related to protein synthesis and muscle fibers adding to accelerated muscle atrophy. Hypoxia induced muscle atrophy has been linked to significant overproduction of inflammatory cytokines that have been shown to inhibit muscle protein synthesis and repair.

Mitochondrial dysfunction and the loss of muscle cell reproduction, and its ability to generate adenosine triphosphate (ATP), the vital energy required for survival, has been identified especially in patients with hypoxia due to peripheral artery disease. Research has shown mitochondrial dysfunction through down regulation of electron transport chain complexes within the mitochondria in skeletal muscle compared to match controls.

When oxygen is present, mitochondria theoretically produce aerobically 38 ATP molecules, but under anaerobic conditions and where oxygen is not available, utilizing the same raw materials glycolysis produces only 2 ATP molecules.

The many unique properties of Ox66™ makes it therapeutically beneficial to supply hypoxic skeletal muscle cells such as mitochondria when administered to a mammal, such as by IV administration shown in FIG. 1. First, Ox66™ is uniquely suited to offload bioavailable oxygen gas (O₂) to increase tissue oxygen tension and availability that results in improved mitochondrial function and efficiency. The bioavailable O₂ molecules are 100% pure oxygen, with the partial pressure of the oxygen is 760 mmHg. This is significant, because it highlights how much oxygen is packed into the clathrates. Note that atmospheric oxygen is 21% or 150 mmHg.

The increased oxygen gas (O₂) to skeletal muscle cells when introduced prior to the onset of muscle loss prevents or mitigates the development and/or the progression of sarcopenia and ensuing development of frailty syndrome and loss of function. Second, the ability to deliver oxygen gas O₂ to the mitochondria is extraordinary. The Ox66™ clathrate structure provides a novel delivery structure to provide oxygen gas to the body.

The immediacy of oxygen is physiologically apparent when a metabolic deficiency arises. Situations of pulmonary dysfunction and circulatory failure—hemorrhage, vascular occlusion, cardiac dysfunction etc.—can rapidly endanger the critical organs. First-line treatment (e.g., restoring gas exchange to damaged alveoli or improving systemic and local blood flow) is usually coupled with supplemental oxygen. This can occur through higher FiO₂, mechanical ventilation, or agents that increase circulatory carrying capacity like blood transfusions. However, chronic disorders and comorbidities that produce mild or localized episodes of hypoxia without overt pain/dysfunction may also benefit from oxygen support.

Localized tissue hypoxia can evade systemic oximetry measurements making detection often reliant on presentation. Its manifestation can be illustrated by the sporadic vaso-occlusive crises (VOC) incurred with sickle cell disease. During VOC, occlusions cause microvascular regions of tissues and organs to undergo repeated ischemia/reperfusion injuries that lead to overproduction of reactive oxygen species (ROS), inflammation, and pain ranging from acute to chronic. While sickle cell is lifelong, and approached as a clinical anemia, chronic conditions such as hypertension, obesity, diabetes are not initially treated as microvascular perfusion deficits. They too cause asymptomatic tissue and organ hypoxia that can cumulate as damage to critical organs like heart and kidneys. Healthy persons and those in the early stages of these comorbidities could benefit from supplemental oxygen in terms of systemic inflammatory and cardiovascular burden. Presently, over-the-counter oxygen supplements are gaseous, which necessitates airway access and a tank for efficacy. A time-release, digestive supplement may provide longer and more accessible supplementation to reduce morbidity and improve quality of life.

### Trial

A randomized, double-blinded, placebo-controlled trial was undertaken to determine the safety of Ox66™ as a daily nutraceutical. The physiological workup included blood, and urinalysis along with biweekly telephone contact and adverse event reporting. Efficacy was expected in oximetry metrics, but study criteria also selected for a hypertensive population which provided insight into Ox66™ as therapeutic potential.

### Study Participants

125 people (49:51; female:male) between the 18-72 (mean age 45±5 yr) years of age were consented and enrolled in the study. Subjects were selected based on response to study announcement and fitting within the inclusion/exclusion criteria listed below. The protocol was executed by Clinical Studies USA, which also received IRB approval and operated in compliance with the NIH guidelines for human subjects research. Compliance was reported as 100%.

Study inclusion/exclusion criteria selected for healthy adults <72 years of age who were not on any medications and had no recent history of drug or alcohol abuse/dependence, injury, or disease. Blood pressure above 180/90 mmHg, fragile blood glucose levels, pregnant or nursing, immobility, inability to follow study protocol, and marijuana. use within the last 3 months were additional exclusion factors.

### Study Protocol

This 30-day nutritional study on safety was double-blinded, placebo-controlled, and randomized. Enrollment into study groups was performed by random lottery and both subjects and investigators were blinded to treatments (which were visually similar). Study subjects were instructed to maintain current eating, drinking, exercise, and sleeping habits throughout the study. Natural changes were accepted. Treatments were taken in the morning with food and missed doses were administered as soon as the subject remembered.

100 subjects received Ox66™ with particles sized between 54 and 212 microns such that the Ox66™ is chlorine free, while 25 received a visually identical placebo. Subjects were instructed to dissolve pre-measured ½ teaspoon (36 mg) packets of white powder into water and completely ingest the solution with food once daily in the morning. Compliance was self-reported via bi-weekly telephone contact. Subjects were initially screened (Day 0) via health questionnaire and clinical workup vitals and arterial bloodwork. Days 1. (Baseline) and 30 (end of study) involved a full physiological workup including vitals, blood (three tubes), and urine. No adverse events were reported.

### Sample Analysis

Vital metrics—blood pressure (standard blood pressure cuff), heart rate, and blood oxygen saturation (clinical pulse oximeter), were collected non-invasively. Arterial blood samples were shipped to a Clinical Trials USA affiliated laboratory for analysis. Measurements included Enzyme-Linked Immunosorbent Assay (human IL-6, albumin, myoglobin; commercial kits, abcam, Waltham, Mass.). Immuno-inhibition Method (Creatine Kinase MB), ADVIA Centar XP Immunoanalyzer (Troponin; Siemens Medical Solutions USA Inc, Malvern, Pa.), BN II Analyzer System (CRP; Siemens), UNISTAT Bilirubinometer (Bilirubin: Reichert Technologies, Depew, N.Y.), Dry Chemistry Analyzer (Creatine), Checkmarx (AST), Alanine Aminotransferase ‘SGPT’ Test (ALT; commercial kit), Homocysteine Assay Kit (homocysteine; commercial kit, abcam), Serum Aluminum (Aluminum), Full Spectral Analysis (Mercury, Cadmium), and Zinc Protoporphyrin Lead). Urine tests included urinalysis (visual and Siemens Multistix Reagent Strips), BUN (Semi-automated Mindray BA 88A), and pregnancy test (commercial human chorionic gonadotrophin dipstick). Blood samples were collected and analyzed in triplicate, then averaged.

### Statistics

Data are expressed as mean standard error of the mean (SEM). Statistical comparisons within (Day 1 vs 30) and between experimental groups were made with an Unpaired, Two-tailed T-test. Significance was taken p<0.05.

Discovery of ATP Production using Ox66™ Bioavailable O₂ Molecules

Subjects meeting inclusion criteria were randomly assigned to placebo (N=25) or Ox66™ (N=100) groups. Sex and age breakdown was 49:51 female:male 45±5 yrs, respectively. Blood work and histories showed the overall population with the range of normal for all study metrics except blood pressure. Both groups were similarly hypertensive Hypertension Stage II) with average blood pressures of approximately 144/83 as shown in Table 1 below.

TABLE 1
Clinical Efficacy

| | Day | Placebo (N = 25) | Ox66 ™ (N = 100) |
|---|---|---|---|
| SBP | 1 | 144 ± 0.7 | 144 ± 0.4 |
| mmHg | 30 | 143 ± 1.0 | 140 ± 0.5*^{β} |
| DBP | 1 | 82 ± 0.7 | 83 ± 0.4 |
| mmHg | 30 | 81 ± 0.6 | 80 ± 0.3* |
| MAP | 1 | 130 ± 0.8 | 131 ± 0.4 |
| mmHg | 30 | 128 ± 0.9 | 126 ± 0.4*^{β} |
| PP | 1 | 62 ± 0.8 | 61 ± 0.5* |
| mmHg | 30 | 62 ± 0.8 | 60 ± 0.5* |
| SO₂ | 1 | 97 ± 0.2 | 97 ± 0.1 |
| % | 30 | 97 ± 0.2 | 98 ± 0.1*^{β} |
| CRP | 1 | 9.0 ± 0.27 | 9.1± 0.15 |
| mg/L | 30 | 9.0 ± 0.27 | 6.9 ± 0.16*^{β} |
| ATP | 1 | 4.4 ± 0.28 | 4.4 ± 0.18 |
| mmol/L | 30 | 4.4 ± 0.26 | 7.4 ± 0.15*^{β} |

SBP—systolic blood pressure, DBP—diastolic blood pressure, MAP—mean arterial pressure, PP—pulse pressure, SO₂—% oxygen saturation of hemoglobin, CRP—C-reactive protein, ATP adenosine triphosphate.

Clinical Efficacy. Tabulated values are inclusive of statistically different measurements. Indicators of increased oxygenation SO₂ and ATP) correspond to decreases in blood pressure and the inflammatory marker CRP. Data are mean ±SEM,

*p<0.05 vs Day 1 (BL)

βp<0.05 vs Placebo (Control)

As shown in Table 1. ATP increased from 4.4±0.18 to 7.4±0.15*β, which is a very significant improvement of ATP by 40% over 30 days, which shows a synergy where a small shift toward oxidative phosphorylation restored dysfunctioning mitochondria to the point where oxidative efficiency was also improved. This is due at least because Ox66™ has bioavailable oxygen. This is different that an oxygenated solution where there is no free bioavailable oxygen. in addition, Table 1 shows generation of ATP in healthy adult humans. Digestion of Q66™ creates a longer duration, time-release effect.

It is of note that no significant vasoactive responses were detected using the Ox66™, which contrasts with other oxygen therapeutics such as hemoglobin-based oxygen carriers. This may be due to the oral administration route employed here, meaning only the oxygen, not the clathrate, entered the bloodstream, as well as Ox66™s inorganic S inlet LI re versus purified hemoglobin, which is a known scavenger of the vasodilator nitric, oxide.

### Oximetry, Blood Pressure, and Inflammation

Significant changes were detected in the Ox66™ group for some cardiovascular and inflammatory metrics. On Day 30, Ox66™SO₂ had risen by 1%, which put it higher than placebo and BL. Blood pressure also improved slightly with the systolic pressure dropping by 4 mmHg and the diastolic pressure by 3 mmHg. CRP, a gauge of active inflammation, also decreased by 25%, whereas placebo showed zero change. ATP levels were also significantly elevated following 30 days of Ox66™ as shown in Table 1.

### Safety

Administration of both placebo and Ox66™ was well-tolerated over 30 days. No adverse events were reported and among the study population, compliance was 100%. Day 30 vital, blood, and urine analysis showed both groups remaining within the range of normal for every metric except blood pressure as shown in Table 2 below.

TABLE 2
Safety Profile

| | Day | Placebo (N = 25) | Ox66 ™ (N = 100) |
|---|---|---|---|
| IL-6 | 1 | 1.89 ± 0.03 | 1.89 ± 0.01 |
| pg/mL | 30 | 1.89 ± 0.03 | 1.89 ± 0.01 |
| Bilirubin | 1 | Neg (100%) | Neg (100%) |
| mg/dL | 30 | Neg (100%) | Neg (100%) |
| AST | 1 | 22.6 ± 1.4 | 22.8 ± 0.8 |
| IU/L | 30 | 22.8 ± 1.4 | 22.6 ± 0.8 |
| ALT | 1 | 30.9 ± 0.2 | 28.1 ± 1.1 |
| IU/L | 30 | 31.8 ± 2.2 | 27.8 ± 1.1 |
| Homocysteine | 1 | 15.4 ± 0.5 | 15.4 ± 0.2 |
| mmoles/L | 30 | 15.6 ± 0.5 | 15.4 ± 0.2 |
| Creatine | 1 | 1.1 | 0.97 |
| mg/dL | 30 | 1.1 | 1.1 |
| Troponin | 1 | 0.02 ± 0.00 | 0.02 ± 0.00 |
| ng/mL | 30 | 0.02 ± 0.00 | 0.02 ± 0.00 |
| Creatine Kinase MB | 1 | 2.3 ± 0.14 | 2.2 ± 0.07 |
| ng/mL | 30 | 2.2 ± 0.11 | 2.6 ± 0.06 |
| Myoglobin | 1 | 48 ± 2.7 | 47 ± 1.3 |
| ng/mL | 30 | 48 ± 2.4 | 48 ± 1.2 |
| BUN | 1 | 13.2 ± 0.8 | 12.3 ± 0.4 |
| mg/dL | 30 | 13.3 ± 0.7 | 11.8 ± 0.3 |
| Albumin | 1 | Neg (100%) | Neg (100%) |
| mg/dL | 30 | Neg (100%) | Neg (100%) |
| RBC | 1 | 2.9 ± 0.22 | 3.1 ± 0.1 |
| HPF | 30 | 3.0 ± 0.19 | 2.9 ± 0.12 |
| WBC | 1 | 1.8 ± 0.22 | 1.7 ± 0.11 |
| HPF | 30 | 1.8 ± 0.21 | 1.5 ± 0.11 |

### Safety Data

Included data did not significantly change longitudinally or between groups. Tests that provided categorical data (positive or negative) are accompanied by a percentage of those results. Thus Neg (100%) indicates 100% of samples were negative. Data are mean ±SEM.

Thirty days of daily ingestion were well-tolerated for both intra- and inter-group comparisons against placebo. Improvements in oxygen saturation and inflammatory metrics demonstrated both utility and, in the context of hypertension, therapeutic potential.

The intention was for an observational study in healthy human subjects but due to the inclusion criteria of “no current medications,” the study population (mean age: 45±5 years) was biased for subjects with untreated hypertension. This provided an unexpected look at Ox66™'s therapeutic potential. Ox66™ has no specific vasoactive components, and, per the lack of change in serum aluminum, only oxygen appeared to transit from the gastrointestinal tract to be circulatory system. Surprisingly, this oxygen transfer (1% rise in SO₂) was associated with a blood pressure drop of 4 mmHg was noted for subjects receiving Ox66™.

Ox66™ improvements in SO₂ and CRP show the hypertensive benefit. Likely, hypertensive patients were experiencing asymptomatic regions of tissue or organ ischemia which then responded to the higher oxygen saturation in the Ox66™ group. Relief of hypoxia has the dual effect of lowering hot zones of inflammation and the stress on the cardiovascular system to increase perfusion to ischemic regions.

Systemic hypoxemia was not detected at any point in the study, but a hallmark of hypertension is capillary rarefaction, which exacerbates hypertension by increasing vascular resistance and redistributing blood flow. Rarefaction also creates pockets of tissue and organ ischemia that are associated with the inflammation in chronic kidney disease, which is a complication of hypertension. Before subacute damage progresses to organ dysfunction however, inflammation is detectable in small elevations of serum CRP that correlate to blood pressure. CRP began on the high end of normal for this study (normal is <10 mg/L), which was consistent with uncomplicated hypertension. The 30-day treatment with ^{Ox}66™, which raised SO₂ by 1%, caused an impressive 24% reduction in CRP, while placebo was completely unaffected. Also, IL-6, primarily an indicator of pathogen-induced inflammation, did not change for either group. While it is not possible to say whether Ox66™ had a specific effect on rarefacted tissues, the increased oxygen gradient from the remaining vasculature is a strong candidate for improving the symptoms of rarefaction.

ATP production is indicative of improved mitochondria function, which supports the rarefacted tissue hypothesis. Typically, tissue respiration relies on ATP derived from one of two pathways: glycolysis and oxidative phosphorylation. Glycolosis extracts two ATP molecules from one molecule of glucose under ischemic/hypoxic conditions. When bioavailable oxygen is available, 30-36 ATP are produced from each glucose. The advantage to respiratory efficiency is clear, but not always feasible. Oxidative phosphorylation is continually dependent on oxygen from the blood, while glycolysis can function on cellular stores alone. This permits brief, but high metabolic output when oxygen needs are exceeded, or, chronic, low-powered function during prolonged hypoxia. In the case of rarefacted tissues, the chronic metabolic shift to glycolysis creates acidic conditions and the high potential for mitochondrial dysfunction. This study's increased SO₂ shows a connection between bioavailable oxygen and significant ATP production. As shown in Table 1, ATP increased by 40% over 30 days, which shows a synergy where a small shift toward oxidative phosphorylation restored dysfunctioning mitochondria to the point where oxidative efficiency was also improved.

In the first randomized, double-blinded, placebo-controlled trial, dietary Ox66™ was shown both safe and effective over 30 days of self-administration. Slow, digestive absorption of O₂ molecules increased SO₂ and ATP production, which was associated with lower blood pressure and serum CRP. These findings show that. Ox66™ is an effective and accessible oxygen supplement. Additionally, the blood pressure and inflammatory marker drop in the context of the hypertensive study population warrants further investigation of Ox66™s therapeutic potential.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

I claim:

1. A method of treating mammal, comprising:
administering a therapeutically effective amount a poly-oxygenated aluminum hydroxide composition to the mammal to produce adenosine triphosphate (ATP) mammal, wherein the poly-oxygenated aluminum hydroxide composition comprises a clathrate containing free oxygen gas (O₂) molecules.

2. The method as specified in claim 1, wherein the pool oxygenated aluminum hydroxide composition provides bioavailable O₂ molecules.

3. The method as specified in claim 1, wherein the poly-oxygenated aluminum hydroxide composition contains pure (100%) O₂ molecules.

4. The method as specified in claim 2, wherein the bioavailable O₂ molecules of the poly-oxygenated aluminum hydroxide composition is time released to the mammal.

5. The method as specified in claim 1, wherein the poly-oxygenated aluminum hydroxide composition is administered orally to the mammal.

6. The method as specified in claim 1, wherein the poly-oxygenated aluminum hydroxide composition increases SO₂ in the mammal,

7. The method as specified in claim 1, wherein the mammal is a human.

8. The method as specified in claim 1, wherein the poly-oxygenated aluminum hydroxide composition has particles sized between 54 and 212 microns.

9. A method of a mammal, comprising:
administering a therapeutically effective amount of a poly-oxygenated aluminum hydroxide composition to the mammal to improve mitochondrial function and efficiency, wherein the poly-oxygenated aluminum hydroxide composition composes a clathrate containing pure (100%) oxygen gas (O₂) molecules.

10. The method as specified in claim 9, wherein the poly-oxygenated aluminum hydroxide composition generates adenosine triphosphate (ATP) in the mammal.

11. The method as specified in claim 9, wherein the poly-oxygenated aluminum hydroxide composition provides bioavailable O₂ molecules.

12. The method as specified in claim 11, wherein the bioavailable O₂ molecules of the poly-oxygenated aluminum hydroxide composition is time released to the mammal.

13. The method as specified in claim 9, wherein the poly-oxygenated aluminum hydroxide composition is administered orally to the mammal,

14. The method as specified in claim 9, wherein the poly-oxygenated aluminum hydroxide composition increases SO₂ in the mammal.

15. The method as specified in claim 9, wherein the mammal is a human.

16. The method as specified in claim 9, wherein the poly-oxygenated aluminum hydroxide composition has particles sized between 54 and 212 microns.
